(19) United States
(12) Patent Application Publication
(10) Pub. No.: US 2010/0172888 A1
(43) Pub. Date: Jul. 8, 2010

(54) HCV-REACTIVE T CELL RECEPTORS

(76) Inventors: Michael I. Nishimura, Chicago, IL (US); Hugo R. Rosen, Portland, OR (US)

Correspondence Address: MICHAEL BEST & FRIEDRICH LLP, 100 East Wisconsin Avenue, Suite 3300, Milwaukee, WI, 53202, US

(21) Appl. No.: 12/092,522

(22) PCT Filed: Nov. 9, 2006
(86) PCT No.: PCT/US06/60697

§ 371 Date: May 2, 2008

Related U.S. Application Data
(60) Provisional application No. 60/735,699, filed on Nov. 10, 2005.

(51) Int. Cl. A61K 35/12; C12N 5/10; C07H 21/04; C12N 15/63; A61P 35/04; C12P 21/06; C12N 5/00

(52) U.S. Cl. 424/93.71; 435/325; 536/23.5; 435/320.1; 435/455; 435/69.1

(57) ABSTRACT

Provided are cells expressing HCV epitope-reactive recombinant T cell receptors useful in the treatment and/or prevention of acute or chronic HCV and HCV-related conditions or malignancies. The invention further provides methods of preparing HCV epitope-reactive T cell receptors and methods of treatment using cells expressing HCV epitope-reactive recombinant T cell receptors. Polynucleotides, constructs and vectors encoding HCV epitope-reactive recombinant T cell receptors are also provided.

### CROSS REFERENCE TO RELATED APPLICATIONS

This application claims benefit of priority from U.S. Provisional Application Ser. No. 60/735,699, filed Nov. 10, 2005, and incorporated herein by reference in its entirety.

### STATEMENT REGARDING FEDERALLY SPONSORED RESEARCH OR DEVELOPMENT

This invention was made with United States government support under CA90873, CA102280, CA100240 and R01 DK060590 awarded by the National Institutes of Health. The U.S. government has certain rights in this invention.

### NAMES OF PARTIES TO A JOINT RESEARCH AGREEMENT

Not applicable.

### REFERENCE TO A SEQUENCE LISTING

This application includes a sequence listing submitted herewith. The content of the sequence listing is incorporated herein by reference in its entirety.

### INTRODUCTION

1. Field of the Invention

The present invention relates generally to the field of immunology and immune-mediated therapies. More particularly, the invention pertains to production and use of cells expressing a recombinant hepatitis C virus-reactive T cell receptor. Such cells are able to mediate an immune response in the recipient which may be effective in reducing viral titer and/or clearing the virus, as well as in treating HCV-related conditions such as cirrhosis and hepatocellular carcinoma.

2. Background

Hepatitis C is a viral infection of the liver which was previously referred to as parenterally transmitted “non-A, non-B hepatitis” until identification of the causative agent in 1989. The discovery and characterization of the hepatitis C virus (HCV) led to the understanding of its primary role in post-transfusion hepatitis and its tendency to induce persistent infection.

HCV is a major cause of acute hepatitis and chronic liver disease, including cirrhosis and hepatocellular carcinoma. Globally, an estimated 170 million persons are chronically infected with HCV, and 3 to 4 million persons are newly infected each year. It is estimated that over 3% of the worldwide population harbors chronic HCV infections.

The standard anti-viral therapy for HCV infection is interferon-α in combination with ribavarin. However, many patients fail to respond to this therapy, which is also associated with significant side effects. Clearly, more effective therapies for HCV infected patients are necessary in order to reduce the worldwide morbidity and mortality from HCV infection and HCV-related malignancies.

There is evidence that the immune system can mediate clearance of HCV infection. Despite the fact that HCV reactive T cells have been isolated which recognize more than fifty antigenic HCV epitopes, a majority of patients exposed to HCV develop chronic infection. The development of chronic infection is influenced, at least in part, by the tendency of the virus to rapidly mutate, leading to antigen escape variants. Moreover, it has been speculated that both low T cell avidity and an ineffective cytokine profile generated in response to infection may contribute to the development of chronic infection rather than viral clearance.

### SUMMARY OF THE INVENTION

The present inventors have previously shown that HCV-positive liver transplant patients that received HLA-disparate liver allografts have HCV-reactive T cells of host origin that are restricted by the donor HLA molecules. Initial studies of these T cells showed that they have relatively high affinity for their HCV epitope ligand. This work was extended, and the inventors subsequently cloned the T cell receptors from the HCV epitope reactive T cells and developed a vector to deliver the T cell receptor coding sequences to cells, e.g., Peripheral Blood Lymphocyte (PBL)-derived T cells or hematopoietic stem cells, ex vivo. The engineered autologous cells are returned to the HCV-infected patient to effect treatment of acute or chronic HCV infection or HCV-related malignancies. Unlike vaccine and peptide/MHC tetramer strategies, this approach does not rely on the patient's T cell receptor repertoire and/or precursor frequency. Moreover, in some embodiments of the invention, cells which natively express an HCV epitope-reactive T cell receptor can be engineered to express a second, recombinant T cell receptor which is reactive to a different HCV epitope, thereby diminishing the impact of HCV escape variants on chronic infection.

Accordingly, in one aspect, the invention provides a cell having an HCV epitope-reactive recombinant T cell receptor.

In another aspect, the invention provides an isolated polynucleotide that includes a sequence encoding an α-chain of an HCV epitope-reactive T cell receptor having at least 95% sequence identity to SEQ ID NO:2 of the sequence listing. The invention also provides an isolated polynucleotide that includes a sequence encoding a β-chain of a HCV epitope-reactive T cell receptor having at least 95% sequence identity to SEQ ID NO:4 of the sequence listing. In further aspects, the invention provides an isolated polynucleotide that includes a sequence encoding an α-chain of an HCV epitope-reactive T cell receptor having at least 95% sequence identity to SEQ ID NO:2 and a sequence encoding a β-chain of an HCV epitope-reactive T cell receptor having at least 95% sequence identity to SEQ ID NO:4. The invention also provides constructs and vectors that include the isolated polynucleotides.

In an additional aspect, the invention provides a method of preparing an HCV-reactive T cell for delivery to a subject. The method includes a step of transducing a T cell isolated from the subject with a construct of the invention, as described above.

In yet another aspect, the invention provides a method of preparing an HCV-reactive T cell receptor. The steps of the method include: (a) isolating an HCV-reactive T cell from a HCV-positive recipient of an HLA mismatched liver allograft or from an HCV-exposed aviremic individual; (b) cloning a polynucleotide sequence encoding the α- and β-chains of the T cell receptor from the HCV reactive T cell; (c) delivering the polynucleotide sequence of step (b) to a cell; and (d) incubating the cell under conditions suitable for expression of the T cell receptor by the cell.

In a further aspect, the invention provides a method of treating an HCV-infected subject or inhibiting reactivation of an HCV infection in a subject. The method includes a step of administering to the subject an immunotherapeutically effective amount of cells comprising an HCV epitope-reactive recombinant T cell receptor.

In still another aspect, the invention provides a method of treating HCV-related hepatocellular carcinoma in a subject. The method includes a step of administering to the subject an immunotherapeutically effective amount of cells comprising an HCV epitope-reactive recombinant T cell receptor.

In a further aspect, the invention provides for use of a cell comprising an HCV epitope-reactive recombinant T cell receptor in the preparation of a medicament for treating HCV infection or HCV-related hepatocellular carcinoma.

### BRIEF DESCRIPTION OF THE DRAWINGS

FIG. 1 depicts FAGS analysis of peripheral blood mononuclear cells (PBMC) from HLA-A2⁻ patients that had received an HLA-A2⁺ liver allograft which were stained with anti-CD8 and HLA-A2 tetramers loaded with the indicated HCV peptides. The percentage of double-positive cells is indicated in the upper right hand corner.

FIG. 2 is a graph showing the amount of IFN-γ released from four T cell clones after stimulation with the indicated cell transduced with HCV NS3:1406-1415 or an empty vector.

FIG. 3 depicts retroviral vector constructs encoding HCV TCR and CD8.

FIG. 4 depicts FACS analysis of transduced and untransduced SupT1 cells stained with anti-CD3 and anti-TCR antibodies.

FIG. 5 is a graph showing the amount of IL-2 released from untransduced or transduced Jurkat cells after treatment with T2 cells alone, lonomycin, T2 cells loaded with HCV NS3:1406-1415, T2 cells loaded with a CMV peptide and T2 cells loaded with a tyrosinase peptide.

FIG. 6A is a graph showing the amount of IFN-γ released by the parent T cell clones in response to stimulation with T2 cells loaded with increasing concentrations of HCV peptide.

FIG. 6B is a graph showing the amount of IL-2 released by Jurkat cells transduced with the HCV TCR in response to stimulation with T2 cells loaded with increasing concentrations of HCV peptide.

FIG. 7 is a graph showing the amount of IL-2 released by transduced and untransduced Jurkat cells in response to HLA-A2⁺ and HLA-AZ cell lines expressing either HCV or CMV peptides.

FIG. 8 depicts FACS analysis of untransduced, HCV TCR transduced and HCV TCR CD8 transduced Jurkat cells stained with anti-CD8 antibody (D, E, F) or HLA-A2/HCV NS3:1406-1415 tetramers (A, B, C).

FIG. 9 is a graph showing the amount of IL-2 released by HCV TCR transduced Jurkat cells as compared to HCV TCR, CD8 transduced Jurkat cells in response to decreasing concentrations of the HCV peptide.

FIG. 10 is a graph showing the amount of IFN-γ released from normal peripheral blood-derived T cells transduced with the HCV TCR (Donor B, D, and F) after stimulation with T2 cells alone or T2 cells loaded with irrelevant (CMV) or HCV peptide, or HLA-A2⁺ cells (624 MEL and RCC UOK131 cells) loaded with irrelevant or HCV peptide.

FIG. 11 is a graph showing the amount of IL-2 released from normal peripheral blood-derived T cells transduced with the HCV TCR (Donor B, D, and F) after stimulation with T2 cells loaded with decreasing concentrations of HCV NS3:1406-1415 peptide as compared to the parent T cell clone.

FIG. 12 is a set of graphs showing the amount of IFN-γ released from normal peripheral blood-derived T cells transduced with the HCV TCR (Donor B, D, and F) and FACS sorted into CD4⁺ and CD8⁺ populations. The cells were stimulated with T2 cells alone or T2 cells loaded with irrelevant (CMV) or HCV peptide, or HLA-A2⁺ cells (624 MEL and RCC UOK131 cells) loaded with irrelevant or HCV peptide.

FIG. 13 is a graph showing the amount of IL-2 released from normal peripheral blood-derived T cells transduced with the HCV TCR (Donor B, D, and F) and the parent T cell clone after stimulation with T2 cells loaded with HCV NS3:1406-1415 peptides containing the indicated mutations.

FIG. 14 is a graph showing the amount of IFN-γ released from normal peripheral blood-derived T cells stimulated with CMV pp65:495-503 peptide and then transduced with the HCV TCR after stimulation with T2 cells loaded with irrelevant (Flu or MART-1), CMV or HCV peptide, or HLA-A2⁺ cells (624 MEL and RCC UOK131 cells) either alone or loaded with CMV or HCV peptide.

FIG. 15 depicts FACS analysis for IFN-γ and CMV tetramer staining of normal peripheral blood-derived T cells stimulated with CMV pp65:495-503 peptide and then transduced with the HCV TCR (lower panels) or left untransduced (upper panels). The T cells had been stimulated overnight with 624 MEL cells alone (left panels), 624 MEL cells expressing HCV NS3:1406-1415 (middle panels) or 624 MEL cells expressing CMV pp65:495-503. The number in the upper right hand corner is the percentage of double stained cells.

### DETAILED DESCRIPTION OF SEVERAL EMBODIMENTS OF THE INVENTION

In response to the need for new strategies for preventing and treating acute and chronic HCV infections and HCV-related malignancies and other conditions, one embodiment of the invention provides a cell comprising an HCV epitope-reactive recombinant T cell receptor (TCR). Such cells are suitable for use in adoptive transfer protocols to provide a particularly effective mode of treatment. “HCV epitope-reactive” is used herein to refer to a TCR which binds to an HCV epitope in the context of a Major Histocompatibility Complex (MHC) molecule to induce a helper or cytotoxic response in the cell expressing the recombinant TCR. The term “recombinant” is used herein to refer to a TCR which is expressed in a cell by introduction of exogenous coding sequences for a TCR. In some embodiments, the recombinant TCR may be expressed in a cell in which the TCR is either not natively expressed or is expressed at levels that are insufficient to induce a response by the cell or a responder cell upon TCR ligand binding.

HCV-reactive T cell receptors can be prepared by transforming or transducing a suitable cell with one or more polynucleotides encoding functional α- and β-chains that can assemble to form a functional HCV-epitope reactive TCR. Cells of the invention are suitably autologous cells, i.e., they are derived from the subject that will receive the transduced or transformed cells. Most suitably, cells are derived from, e.g., peripheral blood lymphocytes or hematopoietic stem cells of the subject. In some embodiments, the cells are T cells that express a CD4 cell surface marker, a CD8 cell surface marker, both a CD4 and CD8 marker (referred to herein as a “double positive), or neither a CD4 nor CD8 cell surface marker (referred to as a “double negative”). In some embodiments, the cell expressing an HCV-reactive recombinant TCR is a T cell that also natively expresses a TCR. The recombinant TCR may bind the same epitope as the natively expressed TCR, or may bind a different epitope. In other embodiments, cells may be transduced with two different recombinant TCRs, i.e., TCRs which bind two different HCV epitopes.

The inventors have found that the peripheral blood of HCV-positive liver transplant patients receiving HLA disparate liver allografts, as well as HCV-exposed patients who have cleared their viral infections, both provide an excellent source of HCV-reactive T cells expressing high affinity TCRs. Accordingly, in some embodiments, HCV-epitope reactive TCRs can be prepared by isolating an HCV-reactive T cell from a HCV-positive recipient of an HLA-mismatched liver allograft, or from an HCV-exposed aviremic individual, and cloning the polynucleotide sequence encoding the α- and β-chains of the T cell receptor from the HCV reactive T cell. Once these sequences have been cloned using standard methods (for example, as described in Molecular Cloning: A Laboratory Manual, 3d ed., Sambrook and Russell, CSHL Press (2001), incorporated herein by reference) the sequences are delivered to a suitable cell and the cell is incubated under conditions suitable for expression of the TCR by the cell. In some embodiments, the suitable conditions may include standard cell culture. When the TCR is expressed in vitro or ex vivo, the cell expressing the TCR may be evaluated for reactivity with HCV epitopes, among other parameters of interest, as known in the art and as exemplified below. In some protocols, i.e., those wherein the vector is administered to a subject, the TCR may also be expressed in vivo to provide a therapeutic effect in a subject in need thereof, i.e., a subject with an acute or chronic HCV infection or an HCV-associated condition.

A “subject” is a vertebrate, suitably a mammal, more suitably a human. As is appreciated, for purposes of study, the subject is suitably an animal model, e.g., a mouse or rat. It will be appreciated that for animal models, the sequence of the TCR α- and β-chains can be selected based on species. In some cases, transgenic animals expressing human MHC molecules may also be useful in evaluating specific embodiments of the invention.

The recombinant TCRs of the invention are most suitably functional in the cell in which they are expressed. That is, they are functional heterodimers of α and β TCR chains associated with a CD3 complex that recognizes an HCV epitope in the context of a Class I or Class II MHC molecule. In humans, the MHC restriction of an epitope is dependent on the particular Human Leukocyte Antigen (HLA) expressed by the cell presenting the antigen. Recombinant TCRs that recognize HCV epitopes restricted on any HLA type (i.e., HLA-A, HLA-B, HLA-C, HLA-DPA1, HLA-DPB1, HLA-DQA1, HLA-DQB1, HLA-DRA, and HLA-DRB1) may be suitable for use in the present invention. For purposes of study, the recombinant TCR may recognize an HCV epitope in the context of an MHC molecule of a species other than human, e.g., H-2K of mouse.

In particular embodiments, the recombinant TCR recognizes HCV epitopes that are HLA-A2 restricted. It is appreciated that roughly half of the human population is HLA-A2 positive, therefore HLA-A2-restricted TCRs will find widespread therapeutic use as described herein. Moreover, HLA-A2 tetramers have been produced that are well-characterized and are commercially available. Such tetramers are useful in preparing the TCRs of the invention, as is described in the examples.

As noted above, the TCRs of the invention are HCV-epitope reactive. There are over 50 known immunoreactive HCV epitopes. Suitable epitopes may be peptides derived from any HCV protein, including HCV core protein, E1, E2, p7, NS2, NS3, NS4a, NS4b, NS5a and NS5b. In some embodiments, the HCV epitope is a mutant form of one of the HCV peptides listed above. As used herein, a “mutant” or “mutant form” of a TCR epitope is one which has an amino acid sequence that varies from a reference virus-encoded sequence via a substitution, deletion or addition of one or more amino acids, but retains the ability to bind and activate the TCR bound and activated by the non-mutated epitope. As will be appreciated, mutants may be naturally occurring or may be recombinantly or synthetically produced.

In some embodiments, a cell includes a TCR comprising an α-chain having at least 95% amino acid identity to SEQ ID NO:2, which has been determined to be the amino acid sequence for a productively rearranged α-chain (AV38s2/AJ30/AC) of a TCR reactive against HCV epitope NS3: 1406-1415. In further embodiments, the α-chain has at least about 97%, at least about 98%, or at least about 99% identity to SEQ ID NO:2. Suitably, the α-chain includes the consecutive sequence of amino acids shown in SEQ ID NO:2.

In other embodiments, a cell includes a TCR comprising a β-chain having at least 95% amino acid identity to SEQ ID NO:4, which has been determined to be the amino acid sequence for a productively rearranged β-chain (BV11s1/BD2s1/BJ2s7/BC2) of a TCR reactive against HCV epitope NS3: 1406-1415. In further embodiments, the β-chain has at least about 97%, at least about 98%, or at least about 99% identity to SEQ ID NO:4. Suitably, the β-chain includes the consecutive sequence of amino acids shown in SEQ ID NO:4.

In particularly suitable embodiments, the cell includes a TCR comprising an α-chain having at least 95% amino acid identity to SEQ ID NO:2 and a β-chain having at least 95% amino acid identity to SEQ ID NO:4.

Percent identity may be determined using the algorithm of Karlin and Altschul (Proc. Natl. Acad. Sci. 87: 2264-68 (1990), modified Proc. Natl. Acad. Sci. 90: 5873-77 (1993)). Such algorithm is incorporated into the BLASTx program, which may be used to obtain amino acid sequences homologous to a reference polypeptide. As will be appreciated, the invention also encompasses TCR α- or β-chains having amino acid sequences including conservative amino acid substitutions. Such substitutions are well known in the art.

Particularly suitable HCV epitopes are provided herein with reference to HCV strain H77 (GenBank Accession Number M67463), where the position of the defined epitope location relative to the sequence of the H77 protein is indicated. Using this system of designating epitopes, particular non-limiting examples of HCV epitopes and mutants thereof which are reactive with recombinant TCRs that may be generated in accordance with the invention are provided in Table 1 below.

TABLE 1
HCV epitopes and mutants.

| Peptide | Sequence | SEQ ID NO. |
|---|---|---|
| HCV core: 35-44 | YLLPRRGPRL | 5 |
| HCV core: 131-140 | ADLMGYIPLV | 6 |
| NS3: 1073-1081 | CINGVCWTV | 7 |
| NS3: 1406-1415 | KLVALGINAV | 8 |
| NS3: 1406-1415 (V1408L) | KLLALGINAV | 9 |
| NS3: 1406-1415 (A1409T) | KLVTLGINAV | 10 |
| NS3: 1406-1415 (I1412L) | KLVALGLNAV | 11 |
| NS3: 1406-1415 (I1412V) | KLVALGVNAV | 12 |
| NS3: 1406-1415 (I1412N) | KLVALGNNAV | 13 |
| NS3: 1406-1415 (V1408T) | KLTALGINAV | 14 |
| NS3: 1406-1415 | KLSSLGLNSV | 15 |
| (V1408S, A1409S, I1412L, | | |
| A1414S) | | |
| NS5b: 2594-2602 | ALYDVVTKL | 16 |

It has been shown that T cell avidity, defined herein as the ability of a T cell to recognize low levels of antigen, correlates with therapeutic efficacy in adoptive transfer studies. Accordingly, in some embodiments of the invention, T cell clones are characterized for relative avidity using, e.g., IFN-γ release assays, as is standard in the art. In general, “high avidity” T cells are defined as requiring 1 mM or less of stimulatory peptide for T cell activation.

The invention further provides isolated polynucleotides comprising a sequence encoding an α-chain of an HCV epitope-reactive T cell receptor. Suitably, the encoded α-chain sequence has at least 95% sequence identity to SEQ ID NO:2. In a particularly suitable embodiment, the isolated polynucleotide comprises the sequence shown in SEQ ID NO:1.

The invention further provides isolated polynucleotides comprising a sequence encoding a β-chain of an HCV epitope-reactive T cell receptor. Suitably, the β-chain sequence has at least 95% sequence identity to SEQ ID NO:4. In a particularly suitable embodiment, the isolated polynucleotide comprises the sequence shown in SEQ ID NO:3.

A particularly suitable isolated polynucleotide of the invention comprises a sequence encoding an α-chain of an HCV epitope-reactive T cell receptor having at least 95% sequence identity to SEQ ID NO:2 and a sequence encoding a β-chain of an HCV epitope-reactive T cell receptor having at least 95% sequence identity to SEQ ID NO:4.

Further embodiments of the invention provide polynucleotide constructs including any of the above-described isolated polynucleotides. Optionally, the coding sequences for the α- and β-chains of the TCR are operably connected to a promoter functional in the cell. Suitable promoters include constitutive and inducible promoters, and the selection of an appropriate promoter is well within the skill in the art. For example, suitable promoters include, but are not limited to, the retroviral LTR, the SV40 promoter, the CMV promoter and cellular promoters (e.g., the β-actin promoter). The term “operably connected” refers to a functional linkage between regulatory sequences (such as a promoter and/or array of transcription factor binding sites) and a second nucleic acid sequence, wherein the regulatory sequences direct transcription of the nucleic acid corresponding to the second sequence.

Constructs may be delivered to cells in vitro, ex vivo or in vivo using any number of methods known to those of skill in the art. For example, if the cells are in vitro or ex vivo, they may be transformed or transduced according to standard protocols, e.g., those described in Molecular Cloning: A Laboratory Manual, 3d ed., Sambrook and Russell, CSHL Press (2001), incorporated herein by reference. The invention also encompasses delivery of constructs to cells in vivo. Suitable methods of delivery of polynucleotide constructs are known in the art, and include but are not limited to, viral vectors, nanoparticles, gold particles, lipoplexes and polyplexes.

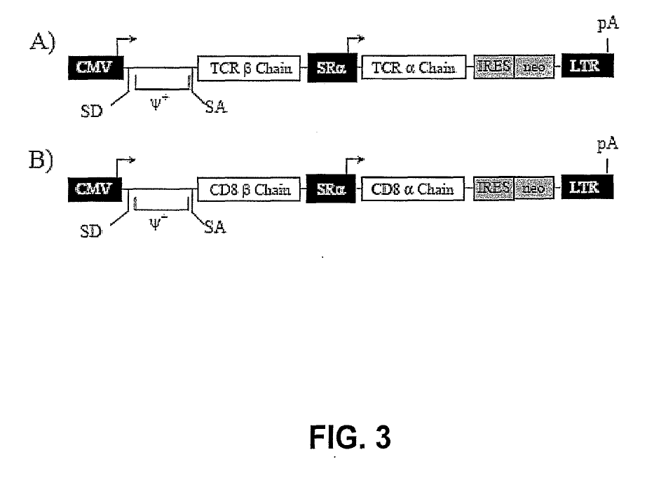

One particularly suitable method of delivery includes use of viral vectors, such as, e.g., lentiviral vectors, retroviral vectors, adenoviral vectors, adeno-associated virus vectors and Herpes Simplex Virus vectors. Retroviral vectors may be particularly suitable for delivery of the constructs either in vitro, ex vivo or in vivo, as described in the examples. One suitable arrangement for a retroviral vector useful in delivering constructs encoding TCRs is shown in FIG. 3.

Vectors comprising polynucleotides encoding TCRs, or cells comprising a recombinant TCR prepared as described above, are suitably administered to a subject to treat an acute or chronic HCV infection or condition (including, e.g., hepatocellular carcinoma) in the subject. In some embodiments, cells expressing recombinant TCRs or vectors comprising polynucleotides encoding TCRs can be prophylactically administered to a subject to inhibit reactivation of an HCV infection.

In some embodiments of the invention, vectors of the invention are administered to cells from a subject ex vivo. Particularly suitable modes of administration of polynucleotide and/or viral vectors will be those that specifically and/or predominantly deliver the TCR coding sequences to T cells and/or hematopoietic stem cells. In the case of a retroviral vector, it is anticipated that suitable dosages will range from about 0.1 μg/10⁶ cells to about 10 μg/10⁶ cells, such as in the range from about 1 μg/10⁶ cells to about 5 μg/10⁶ cells. In particularly preferred embodiments, such dosages will prevent or reduce HCV-related symptoms at least 50% compared to pre-treatment symptoms or compared to a suitable control. It is specifically contemplated that treatment with a retroviral vector of the invention may palliate or alleviate HCV infection or an associated condition, or may reduce incidence of progression to chronic HCV-associated conditions, without providing a cure. In some embodiments, treatment may be used to cure or prevent an acute or chronic HCV infection or an associated condition, including hepatocellular carcinoma.

In some embodiments of the invention, an immunotherapeutically effective amount of cells comprising an HCV epitope-reactive recombinant T cell receptor are administered. As used herein, an “immunotherapeutically effective amount” refers to that amount which results in an immune-mediated prophylactic or therapeutic effect in the subject, i.e., that amount which will prevent or reduce symptoms at least 50% compared to pre-treatment symptoms or compared to a suitable control. The quantity of cells to be administered depends on the subject to be treated, including, e.g., the capacity of the individual's immune system to mount a TCR-mediated immune response, the age, sex and weight of the patient and the severity of the condition being treated. The number of variables in regard to an individual prophylactic or treatment regimen is large, and a considerable range of doses is expected. In general, cells may be administered in an amount from about 5×10⁵ cells/kg body weight to about 1×10¹⁰ cells/kg body weight. More preferably, about 5×10⁶ cells/kg body weight to about 1×10⁸ cells/kg body weight are administered. The maximal dosage of cells or viral vector to be administered to a subject is the highest dosage that does not cause undesirable or intolerable side effects. Suitable regimens for initial administration and additional treatments are also contemplated and may be determined according to conventional protocols.

The following examples are provided to assist in a further understanding of the invention. The particular materials and conditions employed are intended to be further illustrative of the invention and are not limiting upon the reasonable scope of the appended claims.

### Examples

### Example 1

### Isolation of HCV Reactive T Cell Clones

Peripheral blood mononuclear cells (PBMC) were isolated from HLA-A2⁻ patients that had received an HLA-A2⁺ liver allograft. The PBMCs were stained with anti-CD8-FITC and HLA-A2 tetramers loaded with HCV NS3:1073-1081 peptide, HCV NS3:1406-1414 peptide, HCV core:131-139 peptide, HCV NS5:2594-2602 peptide, or an irrelevant peptide (HIV GAG). The tetramers were obtained from Beckman Coulter (Brea, Calif.) or the NIAID tetramer facility.

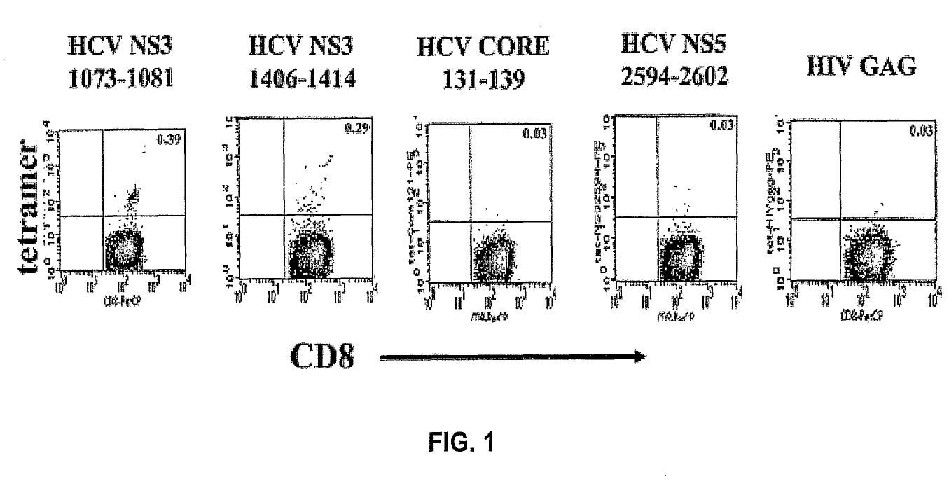

The percentages of stained T cells were determined by FACS analysis using a FACScan flow cytometer (BD Biosciences, Rockville, Md.) and analyzed using CellQuest software (BD Biosciences). The results, depicted in FIG. 1, demonstrate that a significant number of CD8+, HLA-A2-HCV NS3:1073-1081 and HLA-A2-HCV NS3:1406-1415 tetramer-reactive T cells were detected.

Samples with greater than 0.1% HCV tetramer staining T cells were sorted to enrich for HCV reactive T cells and expanded for cloning. Briefly, PBMC were plated into 24-well flat-bottom tissue culture plates at a density of 3×10⁶ cells per well in 2 ml AIM V medium (Invitrogen, Carlsbad, Calif.) supplemented with 10% heat-inactivated pooled human AB serum (Valley Biomedical, Winchester, Va.), 100 U/ml penicillin, 100 μg/ml streptomycin, 2.92 mg/ml L-glutamine, 300 IU/ml recombinant human IL-2 (Chiron Corp., Emeryville, Calif.), and 10 μg/ml peptide.

The cultures were cloned by plating at 10, 3, 1, and 0.3 cells/well in the presence of irradiated PHA stimulated (10 μg/ml) allogeneic PBMC as feeders. Growth positive wells were initially assayed for recognition of peptide loaded T2 cells in IFN-γ release assays. Briefly, T2 cells were obtained from the American Type Culture Collection (Rockford, Md.) and grown in complete medium (CM) consisting of RPMI 1640 medium supplemented with 10% FBS (lnvitrogen Life Technologies, Carlsbad, Calif.), 100 U/mI penicillin, 100 μg/ml streptomycin, and 2.92 mg/ml glutamine. HCV NS3:1406-1415 peptide was obtained from Synthetic Biomolecules (San Diego, Calif.). T cells were co-cultured in a 1:1 ratio with T2 cells loaded with HCV peptides and negative control peptides in a total volume of 200 μl in a 96 well U bottom plate for 18 hours at 37° C. in a humidified incubator as described by Nishimura, et al., Cancer Res. 59:6230-38 (1999), incorporated herein by reference. Supernatants were harvested and the amount of IFN-γ released was measured by ELISA. T cell clones were considered to be antigen reactive when they secreted at least 100 pg/ml IFN-γ per 5×10⁴ T cells in 18 hours and the amount of IFN-γ was at least twice the background levels.

Antigen reactive clones (from plates with fewer than 30% growth positive wells to ensure clonality) were expanded by culturing each clone with irradiated allogeneic PBMC pooled from 3 healthy donors, 30 ng/ml OKT3 (Ortho Biotech, Bridgewater, N.J.) in complete medium containing 10% heat inactivated pooled AB serum, and 200 IU/ml of IL-2 (added every third day of culture).

IFN-γ production by HCV reactive clones was measured using the ELISPOT assay as described by Clay, et al., Clin. Cancer Res. 7:1127-1135 (2001) incorporated herein by reference. 5×10⁴ T cells were co-cultured in a 1:1 ratio with HCV peptide loaded T2 cells overnight in Multiscreen 96 well filtration plates precoated with anti-human IFN-γ mAb. Plates were washed and incubated with a second anti-human IFN-γ mAb conjugated with biotin followed by strepavidin conjugated to alkaline phophatase. Plates were developed using Vectastain AEC substrate and the number of spots in each well were counted using a CTL ELISPOT reader. The number of spots in T cell cultures stimulated with T2 cells pulsed with the relevant HCV peptide epitope were statistically compared (paired T tests) with the number of spots in the same T cell culture stimulated with T2 cells pulsed with an irrelevant control peptide (HIV Pol:476-484). Each T cell culture was also assessed for percentage of cells capable of recognizing processed HCV antigen using HCV⁺ cell lines as stimulators.

The ability of the HCV reactive CTL to lyse HLA-A2⁺, HCV target cells was also measured in ⁵¹Cr release assays as described by Nishimura, et al., J. Immunol. 141:4403-4409 (1988) incorporated herein by reference. Briefly, 10⁶ target cells were labeled for 1 hour at 37° C. with 200 μCi of ⁵¹Cr in CM. 5×10³ labeled targets were incubated with 4×10⁵ (80:1), 1×10⁵ (20:1), 2.5×10⁴ (5:1), and 6.25×10³ (1.25:1) effectors for 4 hours at 37° C. in 200 ml CM. Supernatants were harvested and the amount of ⁵¹Cr released was measured. Total and spontaneous ⁵¹Cr release by each target was determined by incubating 5×10³ labeled target cells in 2% SDS or complete medium respectively for 4 hours at 37° C. Those T cell cultures capable of mediating at least 10% specific lysis at an E:T of 100:1 or less and the observed lysis was at least three times background were considered to be capable of significant specific lysis.

Based on these criteria, four T cell clones reactive to HCV peptide NS3:1406-1414 were obtained. All T cell clones were maintained in RPMI 1640 medium supplemented with 10% heat-inactivated pooled human AB serum, 100 U/ml penicillin, 100 μg/ml streptomycin, 2.92 mg/ml glutamine, and 300 IU/ml recombinant human IL-2 in a 5% CO₂ humidified incubator at 37° C. T cell clones were expanded using 30 ng/ml anti-CD3 mAb (Ortho Biotech, Raritan, N.J.) and 300 IU/ml IL-2 in the presence of irradiated pooled allogeneic PBMC as feeders.

### Example 2

### Recognition of Tumor Cells by T Cell Clones

Melanoma cell lines (MEL) were established from surgical specimens obtained from melanoma patients undergoing immunotherapy at the Surgery Branch, NCI (Topalian, et al., J. Immuno. 142:3714-3725 (1989) and Rivoltini, et al., Cancer Res., 55:3149-3157 (1995), incorporated herein by reference). Renal cell carcinoma lines (RCC) were obtained from patients undergoing radical nephrectomy at the Surgery Branch, NCI (Anglard, et al., Cancer Res. 52:348-356 (1992), incorporated herein by reference). All medium components were obtained from Mediatech (Herndon, Va.) unless noted. MEL 624 (HLA-A2⁺), MEL 624-28 (HLA-A2⁻), RCC UOK131 (HLA A2⁺), and RCC 1764 (HLA-A2⁻) cell lines were maintained in complete medium (CM) consisting of RPMI 1640 medium supplemented with 10% FBS (Invitrogen Life Technologies, Carlsbad, Calif.), 100 U/ml penicillin, 100 μg/ml streptomycin, and 2.92 mg/ml glutamine.

Tumor cell lines engineered to express the HCV NS3:1406-1415 and control epitopes have been described elsewhere (Rosen, et al., J. Immunol. 173:5355-5359 (2004) and Langerman, et al., J. Transl. Med. 2:42 (2004)). Briefly, retroviral vectors containing minigenes encoding the HCV NS3:1406-1415 or control epitopes were used to transduce HLA-A2⁺ and HLA-A2⁻ melanoma (MEL 624 and MEL 624-28, respectively) and renal cell cancer (RCC UOK131 and RCC 1764, respectively) lines. Cells were maintained in RPMI medium as described above supplemented with 500 μg/ml G418 (Research Products International, Mount Prospect, Ill.).

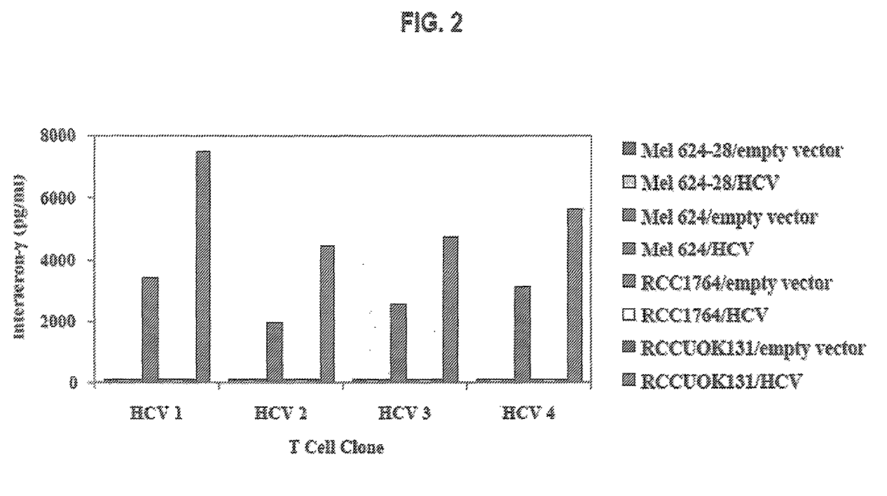

Each T cell clone was co-cultured with melanoma or renal cell carcinoma cells which were HLA-A2⁺ (624 MEL or RCC UOK131) or HLA-A2⁻ (624-628 MEL or RCC 1764). The MEL and RCC cells were transduced with a mini-gene encoding HCV NS3:1406-1415 or the empty vector. The amount of IFN-γ released was measured by ELISA as described above. As shown in FIG. 2, each of the four clones tested specifically secreted IFN-γ when co-cultured with HLA-A2⁺ HCV NS3:1406-1415⁺ targets, but not HCV NS3:1406-1415⁻ targets or HLA-A2⁻ targets.

### Example 3

### TCR α and β Chain Identification

The TCR α chain from each of the four HCV-reactive T cell clones was identified as previously described by Nishimura, at al., J. Immunother. 16:85-94 (1994) and Shilyansky, et al., PNAS 91:2829-2833 (1994), incorporated herein by reference. Briefly, total RNA was isolated from 1-5 million cells using TRIzol (lnvitrogen), and TCR cDNAs were amplified using the 5′ RACE System (Rapid Amplification of cDNA Ends)(Invitrogen) using an a constant region (AC) reverse primer. PCR products were cloned, sequenced, and two productively rearranged α-chains (AV38s2 and AV41s1) were identified.

Both full-length α-chains were amplified from cDNA using AV forward (AV38s2 forward 5′-AAAGTCGACCTGTGAGCATGGCATGCCCTGGCTTCCTG-3′ (SEQ ID NO: 17); AV41s1 forward 5′-AAAGTCGACTAATAATGGTGAAGATCCGGCAATTT-3′ (SEQ ID NO:18)) and AC reverse (5′-AAAGTCGACCCTCAGCTGGACCACAGCCGCAGCGTCATGA GCAGA-3′ (SEQ ID NO:19)) primers containing Sal I restriction sites for subsequent subcloning. PCR products were ligated into the pCR 2.1 TA cloning vector (Invitrogen), and transformed into E. coil TOP 10 competent cells (Invitrogen). Bacterial clones were screened for presence of the α-chain cDNA by PCR, and sequenced to ensure that no errors had occurred during PCR amplification.

The TCR β-chain from the four HCV-reactive T cell clones was identified by RT-PCR using a panel of TCR β-chain V region (BV) degenerate subfamily specific primers as previously described (Anglard, at al., Cancer Res. 52:348-356 (1992)). Total RNA was isolated from 1-5 million cells using TRIzol. First-strand cDNA was synthesized from 1 μg of total RNA using Superscript II reverse transcriptase and oligo(dT)₁₂₋₁₈ (Invitrogen). 10 ng cDNA was PCR-amplified in a 50 μl reaction consisting of 1× PCR buffer, 1.5 mM MgCl₂, 200 μM dNTP, 400 nM TCR BV subfamily-specific forward primer, 400 nM TCR β-chain C region (BC) specific reverse primer, and 1 U Taq DNA polymerase (all PCR reagents: Invitrogen). A BV11 band was obtained from all 4 HCV-reactive T cell clones which was cloned, sequenced, and identified as BV11s1 based on known genomic DNA sequences. The full-length β-chain was amplified from cDNA using forward and reverse primers containing Xho I restriction sites (forward 5′-AAACTCGAGCCCCAACTGTGCCATGACTATC AGGCT-3′ (SEQ ID NO:20); reverse 5′-AAACTCGAGCTAGCCTCTGGAATCCTTTCTCTTG ACCATTGCCAT-3′ (SEQ ID NO:21)), ligated into the pCR 2.1 TA cloning vector, and transformed into E. coli TOP 10 competent cells. Bacterial clones were screened for presence of the β-chain gene, and recombinant clones were sequenced to ensure that no errors had occurred during PCR amplification.

Further DNA sequence analysis revealed that all four T cell clones used the same Jα (AJ30 and AJ49) and Dβ/Jβ (BD2s1/BJ2s7) segments and had identical sequences across the CDR3 region indicating they were sister clones.

### Example 4

### Retroviral Vector Construction and Transduction

The presence of two TCR α chains in these T cell clones necessitated constructing two retroviral vectors to determine which TCR mediated HCV NS3 antigen recognition. The SAMEN CMV/SRα retroviral vector has been previously described (Roszkowski, et al., J. Immunol. 170:2582-2589 (2003), incorporated herein by reference) and was used as the backbone for all retroviral constructs. The HCV TCR α and β chain genes and the CD8 TCR α and β chain genes were inserted into the Xho I and Sal I restriction sites, respectively, of the retrovirus using a rapid ligation strategy to create three retroviral constructs, as shown in FIG. 3. The TCR β chain from HCV clone 3 was first inserted in the upstream cloning site of SAMEN CMV/SRα under the transcriptional control of the MMLV LTR. Then, each of the HCV clone 3 TCR α chains were inserted into the downstream cloning site of SAMEN CMV/SRα under the transcriptional control of the SRα promoter. One retrovirus contained the AV38s2 α chain and the BV11s1 β chain (designated HCV TCR). A second retrovirus contained the AV41s1 α chain and the BV11s1 β chain (designated Alt TCR). A third retrovirus contained the CD8 α and β chains.

Jurkat and SupT1 cell lines (American Type Culture Collection, Rockford, Md.) were maintained in complete medium (CM) consisting of RPMI 1640 medium supplemented with 10% FBS (Invitrogen Life Technologies, Carlsbad, Calif.), 100 U/mI penicillin, 100 μg/ml streptomycin, and 2.92 mg/ml glutamine. 293GP cells were maintained in DMEM supplemented as above.

Retroviral supernatants were prepared using a transient transfection protocol as described by Roszkowski, et al., J. Immunol. 170:2582-2589 (2003). Briefly, 100 cm² tissue culture dishes were coated with 0.02% type B bovine skin gelatin (Sigma-Aldrich, St. Louis, Mo.) in Hanks Basic Salt Solution (HBSS) for 15 minutes at room temperature. 293GP cells were plated at sufficient density to provide 60-70% confluence after 24 hours. Cells were transiently cotransfected with 3 μg retroviral vector and 3 μg plasmid containing the vesicular stomatitis virus envelope gene using Lipofectamine Plus reagents (Invitrogen). Transfection medium was replaced with CM and retroviral supernatants were collected after 24 and 48 hours.

Jurkat and SupT1 cells were transduced by spinoculation as described (Clay, et al., J. Immunol. 163:507-513 (1999)). Briefly, cells were resuspended at 1×10⁶ cells/ml in retroviral supernatant supplemented with 8 μg/ml polybrene (Sigma-Aldrich). Cells were added to 24-well flat-bottom tissue culture plates (1 ml/well), and the plates were centrifuged at 1000×g for 90 minutes at 32° C. Cells were resuspended following spinoculation, incubated for 4 hours at 37° C., and then 1 ml fresh CM was added to each well. This spinoculation procedure was repeated the next day using fresh retroviral supernatant. After 24 hours, transduced cells were selected by adding G418 to each culture (2 mg/ml for Jurkat cells and 2.5 mg/ml for SupT1 cells).

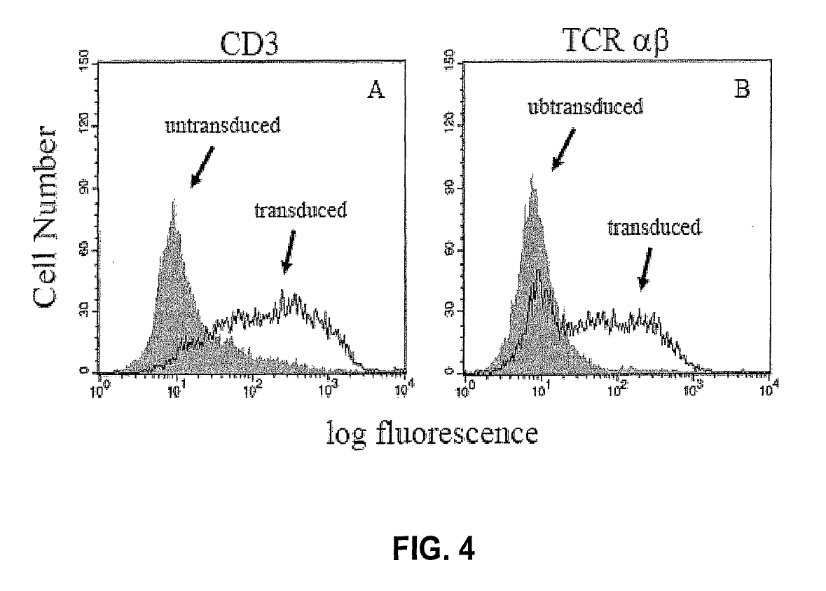

The retroviral vectors (AV38s2/BV11s1 and AV4Is1/BV11s1) were first used to transduce SupT1 cells. SupT1 cells are a CD4⁺/CD8⁺ human T cell lymphoma cell line that does not naturally express CD3 or TCR αβ and was used to validate the expression of cloned TCRs. Transduced and control SupT1 cells were stained with anti-CD-3 and anti-TCR αβ antibodies. As shown in FIG. 4, SupT1 cells transduced with either TCR restored CD3 (FIG. 4A) and TCR αβ (FIG. 4B) expression by FACS analysis (not shown for the AV41s1/BV11s1TCR), indicating both forms of the HCV clone 3 TCR are capable of forming stable TCR/CD3 complexes on the surface of T cells.

### Example 5

### Cytokine Release Assays

Antigen reactivity by the HCV reactive T cell clones and TCR transduced Jurkat cells was measured in cytokine release assays as described above. Briefly, responder and stimulator cells were co-cultured in a 1:1 ratio in 96-well U-bottom tissue culture plates in 200 μl CM. For the Jurkat experiments, 10 ng/ml PMA (Sigma-Aldrich) was added to each well. As a positive control for Jurkat stimulation, maximal cytokine release was obtained by the addition of 1 μg/ml ionomycin (Sigma-Aldrich). Co-cultures were incubated at 37° C. for 20 hours, and then supernatants were harvested. The amount of cytokine released was measured by ELISA using mAbs to IFN-γ (Pierce, Rockford, Ill.) or IL-2 (R&D Systems, Minneapolis, Minn.).

T2 cells were loaded with peptide by incubating 1×10⁶ cells/ml in CM containing varying concentrations of peptide at 37° C. for 2 hours. Peptide-loaded T2 cells were washed with fresh CM prior to co-culture with responders.

To verify the function of the AV38s2/BV11s1 HCV TCR, Jurkat cells were transduced with the HCV TCR retrovirus. Jurkat cells are a CD8⁻ human T cell lymphoma line that expresses its native TCR, therefore, any introduced TCR would have to compete with the endogenous TCR. Furthermore, Jurkat cells expressing a foreign TCR secrete IL-2 upon antigen stimulation in an antigen specific fashion (Roszkowski, et al., Cancer Res. 65:1570-1576 (2005), incorporated herein by reference). Therefore, Jurkat cells represent a model T cell that can be used to evaluate the function of any cloned TCR.

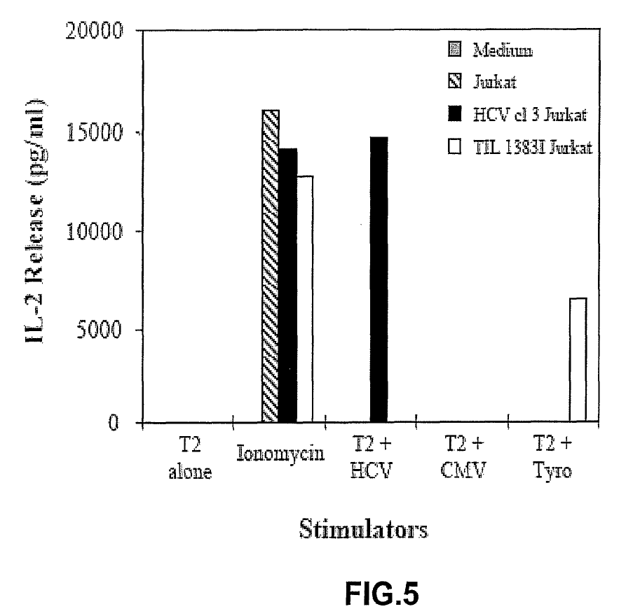

As shown in FIG. 5, Jurkat cells transduced with the HCV TCR secreted significant quantities of IL-2 when stimulated with T2 cells loaded with HCV NS3:1406-1415 peptide. These cells were considered to be HCV reactive since HCV TCR-transduced Jurkat cells did not recognize T2 cells alone or T2 cells loaded with irrelevant peptides (CMV pp65:495-503 (NLVPMVATV) (SEQ ID NO:22) or tyrosinase:368-376 (YMDGTMSQV) (SEQ ID NO:23) Control Jurkat cells transduced with a TCR that mediates HLA-A2 restricted recognition of tyrosinase (TIL) only secreted IL-2 when stimulated with T2 cells loaded with tyrosinase:368-376 and not HCV NS3:1406-1415 or CMV pp65:495-503 (FIG. 5). These experiments demonstrate that HCV NS3:1406-1415 peptide recognition was mediated by the AV38s2/BV11s1 TCR cloned from HCV clone 3 cells.

### Example 6

### Relative Avidity of HCV TCR Transduced Jurkat Cells

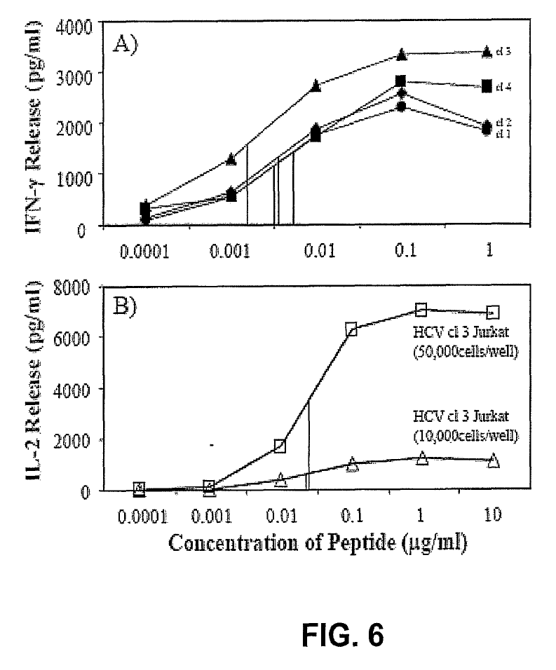

In order to determine the contribution of the AV38s2/BV11s1, clone 3 HCV TCR to the relative avidity of transduced T cells bearing native, non-HCV reactive TCRs, the parental HCV reactive T cell clones and TCR transduced Jurkat cells were stimulated with T2 cells loaded with decreasing amounts of HCV NS3:1406-1415 peptide and the amount of cytokine released was measured by ELISA. As shown in FIG. 6, the parental HCV T cell clones secreted significant quantities of IFN-γ (greater than 100 pg/ml and at least twice background) when stimulated with T2 cells loaded with <1 ng/ml concentrations of peptide and required that T2 cells were loaded with between 1 and 10 ng/ml peptide in order to elicit half maximum production of IFN-γ (FIG. 6A). In contrast, the HCV TCR-transduced Jurkat cells required T2 cells to be loaded with 10 ng/ml or greater concentrations of peptide to elicit significant (greater than 100 pg/ml and at least twice background) IL-2 release (FIG. 6B). The half maximum response was between 20 and 30 ng/ml regardless of the number of HCV TCR transduced Jurkat cells used in the assays. Both the relative avidity and half maximum response for the HCV TCR-transduced Jurkat cells were at least 10-fold lower than the parent T cell clones (Compare FIG. 6A to FIG. 6B). Given the competition with the endogenous TCR chains in Jurkat cells, it was expected that the TCR transduced Jurkat clone would express reduced levels of HCV TCR relative to the parent T cell clone. As a result, the differences in relative avidity were not surprising and were consistent with results obtained with other TCRs (Cole, et al., Cancer Res. 55:748-752 (1995)).

### Example 7

### Recognition of Processed Antigen by HCV TCR Transduced Jurkat Cells

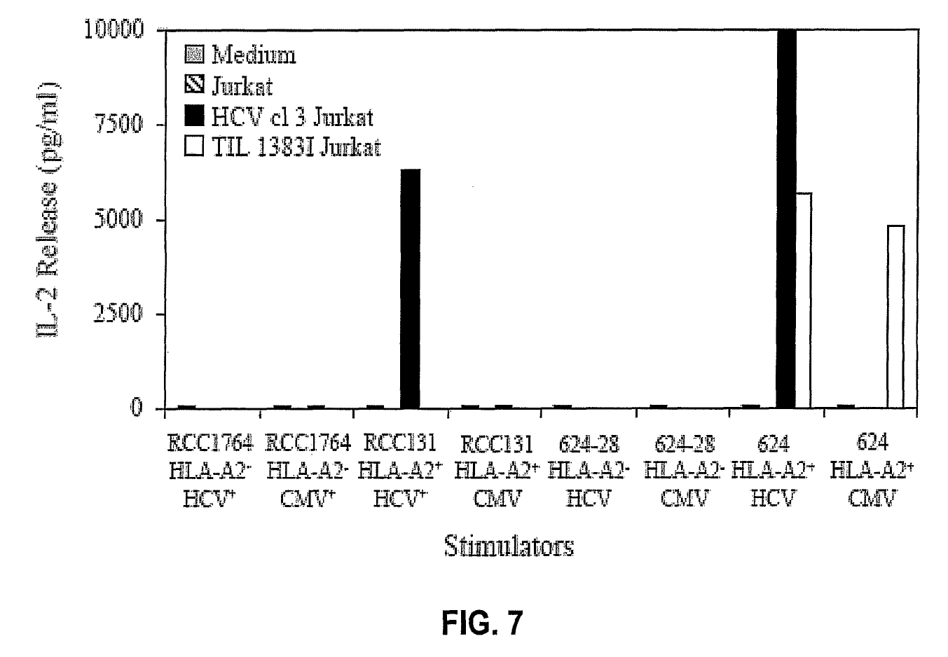

As described above, a panel of HCV⁺ targets, including human melanoma and renal cell carcinoma cells engineered to express the HCV NS3:1406-1415 peptide epitope and parent HCV reactive T cell clones, was used to assess the ability of HCV TCR transduced Jurkat cells to recognize endogenously encoded antigen presented through the MHC class I pathway. As shown in FIG. 7, HCV TCR transduced Jurkat cells secreted significant amounts of IL-2 (greater than 100 pg/ml and at least twice background) when co-cultured with HLA-A2⁺ HCV NS3:1405-1415⁺ but not HLA-A2⁻ HCV NS3:1405-1415⁺ or HLA-A2⁺ CMV pp65:495-503⁺ tumor cells. Control TIL 1383I TCR transduced Jurkat cells secreted IL-2 only when stimulated with HLA-A2⁺ melanoma cells and not with HLA-A2⁻ melanoma cells or renal carcinoma cells. These results indicate that the HCV TCR can transfer the ability to recognize processed antigen to other effector cells. Furthermore, the absence of CD8 expression on Jurkat cells did not preclude the HCV TCR Jurkat cells from recognizing HCV⁺ cells. Therefore, CD8 expression was not required for recognition of processed antigen.

### Example 8

### Role of CD8 in Tetramer and Antigen Recognition

In order to explore further the results obtained in Example 7, and to determine the role of CD8 in stimulating HCV TCR transduced cells, HCV TCR transduced Jurkat cells were transduced to express human CD8. Full length CD8 α and β chains were amplified by RT-PCR from human T cell cDNA. The cloning primers used to amplify the CD8 α (forward 5′-AAACTCGAGCGCGTCATGGCCTTACCAGTGACCG-3′ (SEQ ID NO:24); reverse-5′-AAACTCGAGTTAGACGTATCTCGCCGAAAG-3′ (SEQ ID NO:25)) and β (forward 5′AAAGTCGACGCCACGATGCGGCCGCGGCTGTGGCT-3′ (SEQ ID NO:26); reverse-5′-GTCGACAATAAACACTTCAACAAAGCACTC-3′ (SEQ ID NO:27)) chains contained Xho I or Sal I restriction sites, respectively, for subsequent subcloning. PCR products were ligated into the pCR 2.1 TA cloning vector and transformed into E. coli TOP 10 competent cells. Bacterial clones were screened for presence of the full length CD8 α or β chain genes and recombinant clones were sequenced to ensure that no errors had occurred during PCR amplification.

The cell surface expression of the TCR and other T cell markers was measured by immunofluorescence staining and quantified by flow cytometry as described (Langerman, et al., J. Transl. Med. 2:42 (2004)). The following antibodies were used: anti-CD3-PE, anti-CD8-FITC, anti-TCR α-β-PE (BD Biosciences, San Diego, Calif.), and anti-TCR Vβ11-FITC (Beckman Coulter, Brea, Calif.). The following PE-labeled HLA-A *0201 tetramers were used: HCV NS3:1406-1415 and CMV pp65:495-503 (Beckman Coulter). Flow cytometry was performed using a FACScan flow cytometer (BD Biosciences), and data were analyzed with the CellQuest program (BD Biosciences).

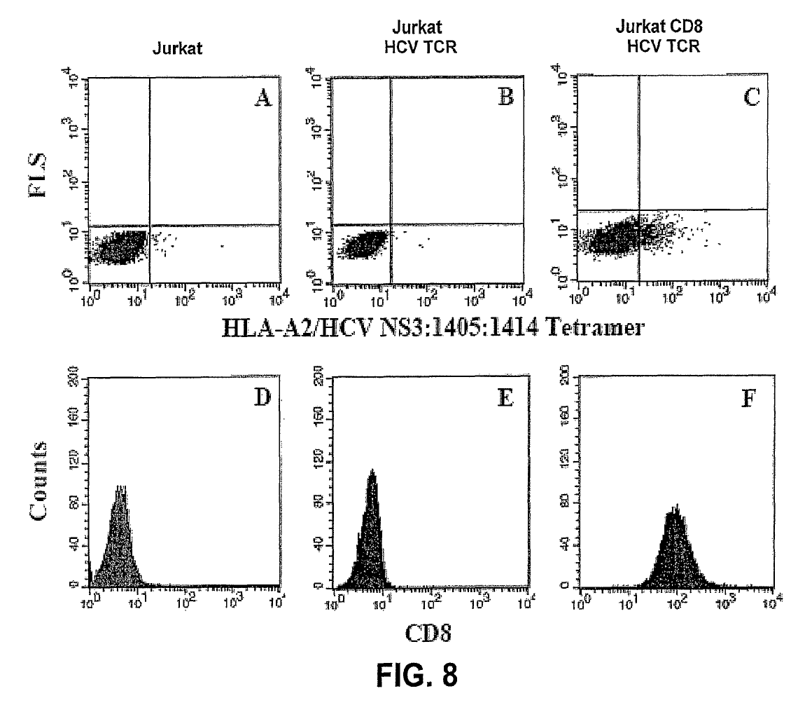

As shown in FIG. 8, untransduced and HCV TCR-transduced Jurkat cells do not express CD8 (FIGS. 8D and 8E) and do not bind tetramers (FIGS. 8A and 8B). In contrast, HCV TCR Jurkat cells transduced with the CD8 retrovirus express high levels of CD8 (FIG. 8F) and some of the cells could bind tetramers (FIG. 8C). When co-cultured with T2 cells loaded with 1 μg/ml HCV NS3:1406-1415 peptide, the CD8⁺ HCV TCR Jurkat cells secreted 53,284 pg/ml IL-2 and CD8⁻ HCV TCR Jurkat cells secreted 30,822 pg/ml IL-2. In contrast, when co-cultured with T2 cells loaded with a control tyrosinase peptide, these cells secreted 88 and 48 pg/ml IL-2, respectively. These results confirm that tetramer binding to the HCV TCR requires CD8 expression and while CD8 expression is not necessary for IL-2 production, the expression of CD8 augments the stimulation of HCV TCR Jurkat cells by HCV⁺ target cells.

### Example 9

### Influence of CD8 on the Avidity of HCV TCR Transduced Jurkat Cells

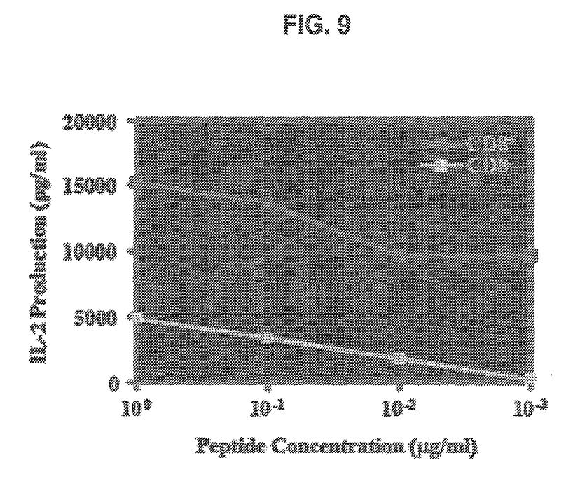

HCV TCR transduced Jurkat cells were transduced to express CD8 in Example 8. The resulting CD8⁺ Jurkat cells were compared to CD8⁻ Jurkat cells for sensitivity to antigen stimulation. HCV TCR transduced Jurkat cells were co-cultured overnight with T2 cells loaded with decreasing amounts of the wild type HCV NS3:1406-1415 peptide. The amount of IL-2 released by 10⁵ cells was measured by ELISA. The average of triplicate wells is shown in FIG. 9. While it is clear that CD8 is not required for efficient antigen recognition, expressing CD8 in Jurkat cells enhances the response to HCV.

### Example 10

### Peripheral Blood T Cells Transduced with the HCV TCR Recognize HCV+ HLA-A2+ Cells

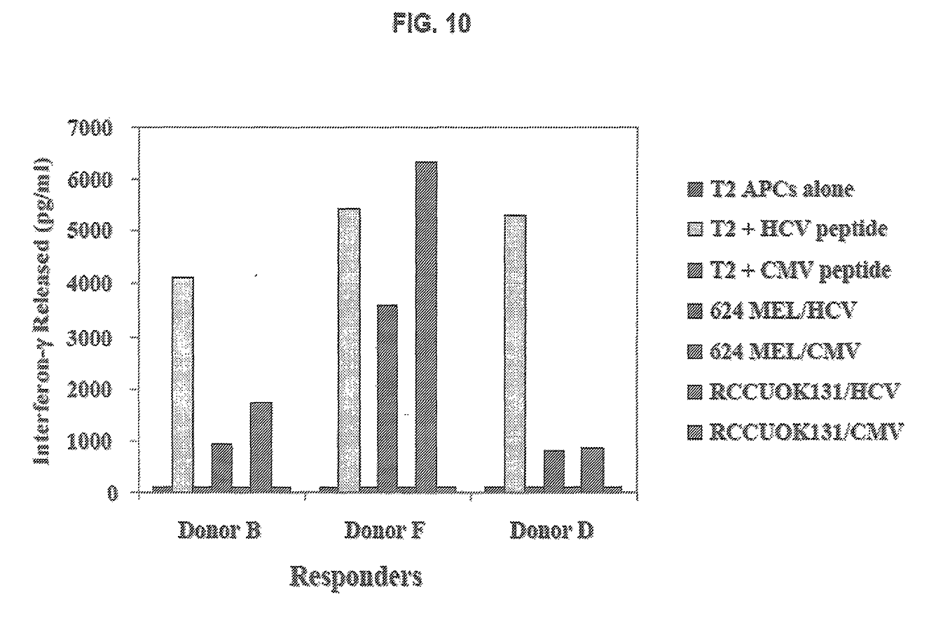

Normal peripheral blood (PBL)-derived T cells were activated with anti-CD3 and IL-2 then transduced to express the HCV TCR as described above. The resulting HCV TCR transduced T cell cultures were assayed for their ability to recognize T2 cells loaded with HCV NS3:1406-1415 peptide and tumor cells engineered to express this peptide epitope. The amount of IFN-γ released was measured by ELISA as described above. The results, shown in FIG. 10, demonstrate that expression of this HCV reactive TCR in normal PBL-derived T cells resulted in recognition of HCV peptide loaded T2 cells and HCV⁺ cell lines, but not cell lines or T2 cells loaded with an irrelevant CMV peptide.

### Example 11

### Avidity of HCV TCR Transduced PBL-Derived-T Cells

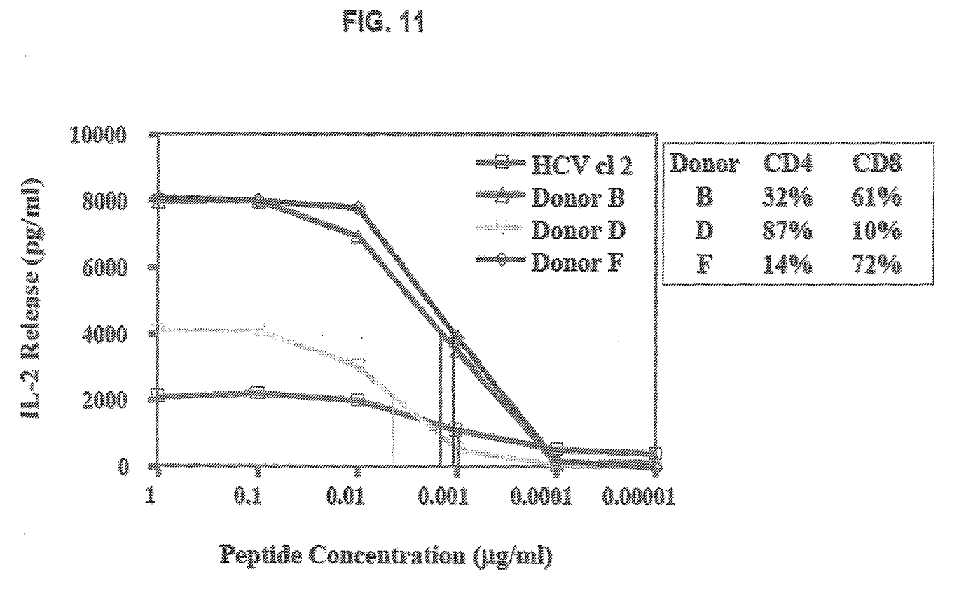

The parent HCV reactive T cell clone and three HCV TCR transduced PBL-derived T cell cultures were co-cultured overnight with T2 cells loaded with decreasing amounts of the wild type HCV NS3:1406-1415 peptide. The amount of interferon-γ released by 10⁵ T cells was measured by ELISA. The average of triplicate wells is shown in FIG. 11. Vertical lines represent the amount of peptide required to elicit half-maximum interferon-γ release. The percentage of CD4 and CD8 T cells in each culture was 0%/100% for the HCV T cell clone, 32%/61% for Donor B, 87%/10% for Donor D, and 14%/72% for Donor F. The results demonstrate that the HCV TCR transduced T cell cultures produced more IFN-γ and had avidity similar to the parent T cell clone.

### Example 12

### Antigen Recognition by HCV TCR Transduced CD4+ and CD8+ T Cells

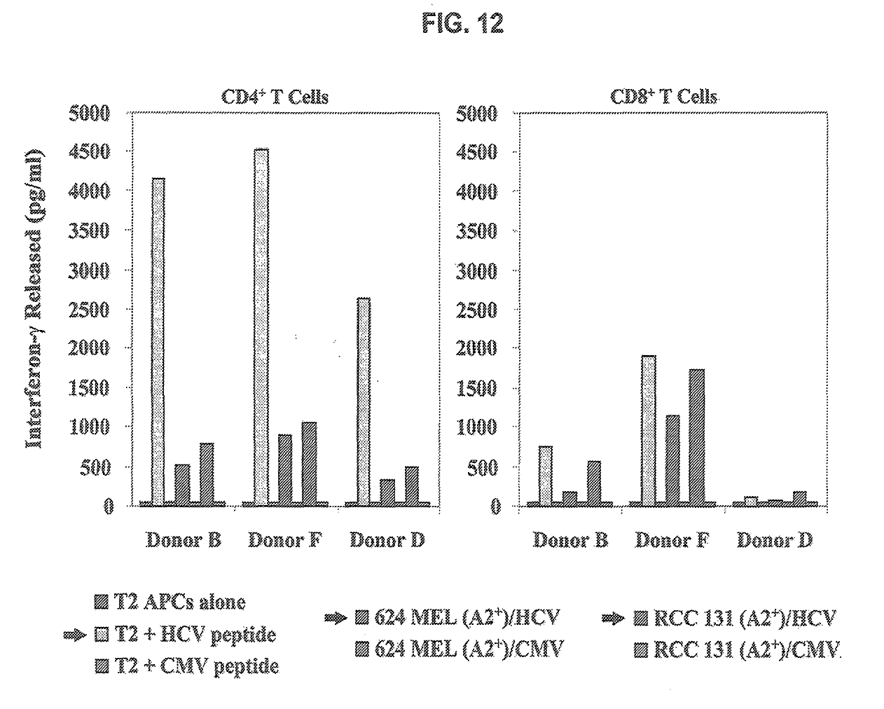

HCV TCR transduced T cells were stained with anti-CD4 or anti-CD8 antibodies and sorted by FACS to obtain cultures that were greater than 99% pure. Purified CD4⁺ and CD8⁺ T cells were co-cultured overnight with T2 cells loaded with 5 μg/ml of HCV or irrelevant CMV peptide (pp65:495-503) or either 624 melanoma cells or renal carcinoma 131 cells expressing the HCV NS3:1406-1415 peptide or the CMV pp65:495-503 peptide. The amount of interferon-γ released by 10⁴ T cells was measured by ELISA. The average and standard deviation of triplicate wells is shown in FIG. 12. The results demonstrate that purified HCV TCR transduced CD4⁺ and CD8⁺ T cells could both recognize HCV peptide loaded T2 cells and HCV⁺ cell lines.

### Example 13

### Recognition of Mutant HCV NS3:1406-1415 Peptides by HCV TCR Gene Modified T Cells

The HCV NS3:1406-1415 peptide sequence was used to scan GenBank for related sequences. Of the 1,000 sequences recovered, eight naturally occurring mutant epitopes were identified as indicated in Table 2 below.

TABLE 2
Mutant HCV NS3: 1406-1415 Peptides.

| Peptide | Sequence | SEQ ID NO. |
|---|---|---|
| HCV NS3: 1406-1415 | KLVALGINAV | 8 |
| V1408L | KLLALGINAV | 9 |
| A1409T | KLVTLGINAV | 10 |
| I1412L | KLVALGLNAV | 11 |
| I1412V | KLVALGVNAV | 12 |
| I1412N | KLVALGNNAV | 13 |
| V1408S, A1409G, I1412L | KLSGLGLNAV | 28 |
| V1408T | KLTALGINAV | 14 |
| V1408S, A1409S, I1412L, | KLSSLGLNSV | 15 |
| A1414S | | |
| Tyrosinase: 368-376 | YMDGTMSQV | 23 |

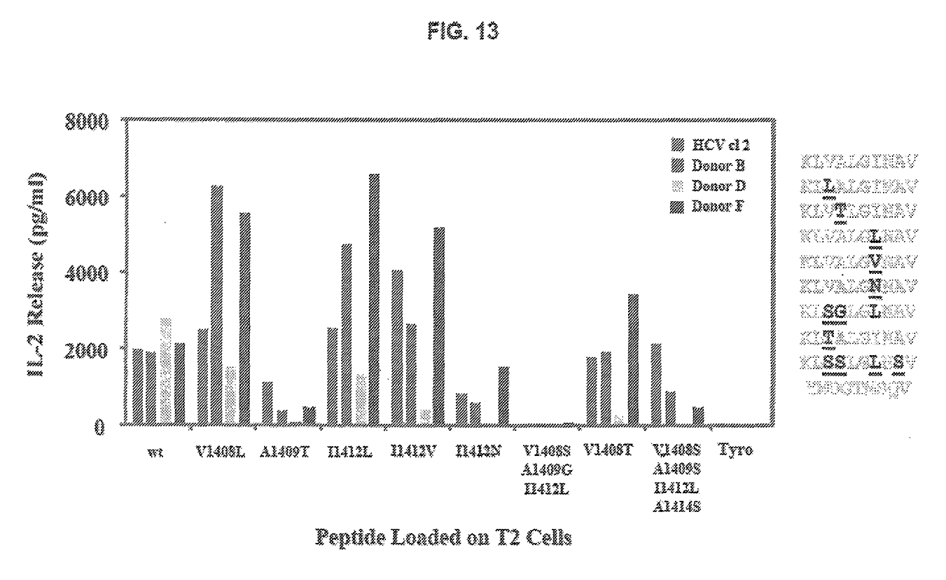

Each of the above peptides were synthesized and used to stimulate HCV TCR transduced T cells. The parent HCV reactive T cell clone and three HCV TCR transduced PBL-derived T cell cultures were co-cultured overnight with T2 cells loaded with 5 μg/ml of the wild type HCV NS3:1406-1415 peptide, the mutant peptides, or a control tyrosinase:365-376 peptide. The amount of interferon-γ released by 10⁵ T cells was measured by ELISA. The average and standard deviation of triplicate wells is shown in FIG. 13. Seven of the eight mutated peptides were recognized by the parent T cell clone and at least two of the HCV TCR transduced T cell cultures. The results show that the HCV TCR recognizes several naturally occurring mutant epitopes and thus may limit the opportunity of HCV to escape immune recognition via mutation of the epitope.

### Example 14

### Antigen Recognition by Bifunctional T Cells

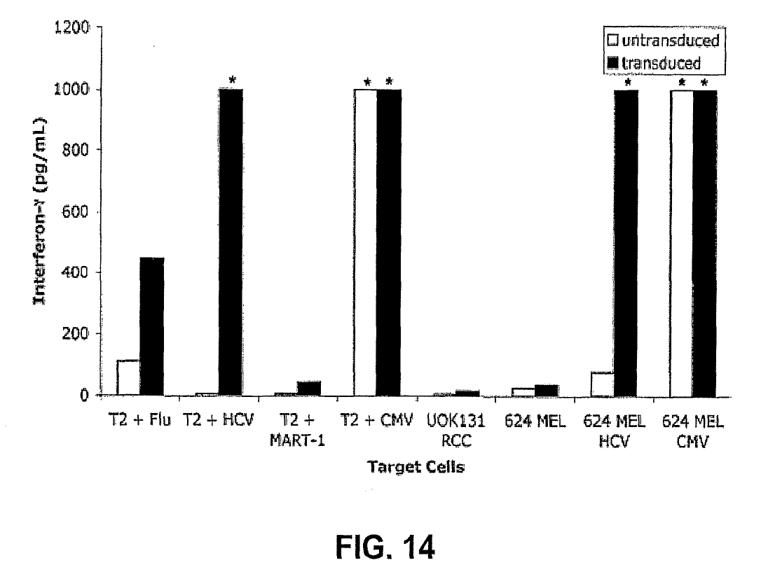

Normal PBL-derived T cells were stimulated with CMV pp65:495-503 peptide and then transduced with the HCV TCR, as described by Heemskerk, et al., Bone Marrow Transpl. 33:S21 (2004) and Langerman, et al., J. Transl. Med. 2:42-49 (2004). PBL-derived T cells from a normal donor were stimulated for three days with 5 μg/mI CMV pp65:495-503 peptide and IL-2 then transduced with a retrovirus encoding the HCV TCR. Bulk cultures were assayed for IFN-γ release when stimulated with peptide loaded T2 cells and melanoma (624) or renal cancer (RCC 131) cells expressing HCV NS3:1406-1415 or CMV pp65:495-503. The amount of interferon-γ released was measured by ELISA. As shown in FIG. 14, the resulting TCR transduced T cell cultures recognized CMV pp65:495-503 or HCV NS3:1406-1415 peptide loaded T2 cells as well as CMV⁺ and HCV⁺ tumor cells.

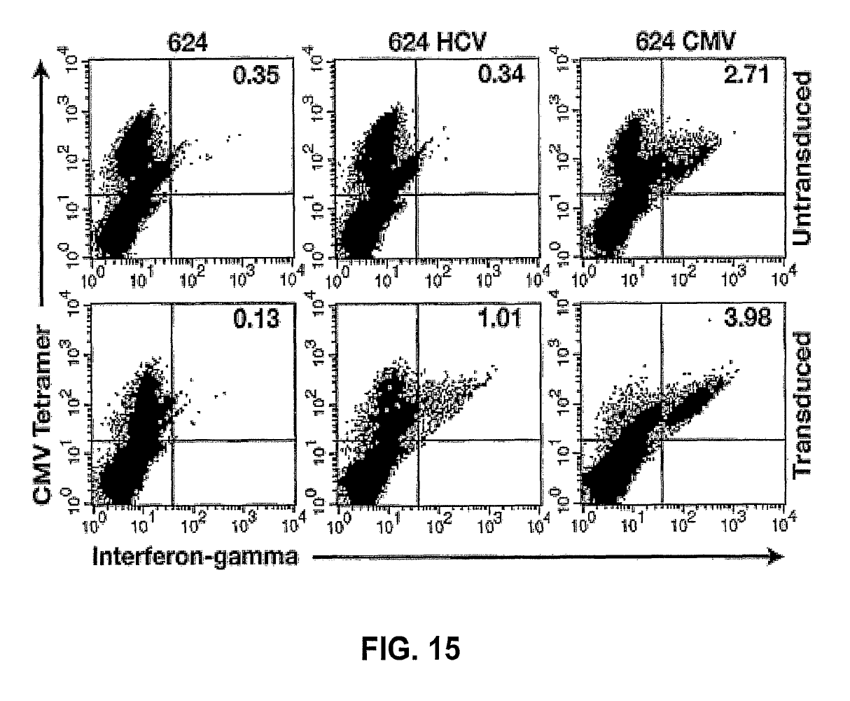

As shown in FIG. 15, the dual recognition of HCV NS3:1406-1415 and CMV pp65:495-503 by the bifunctional T cells was established using a combination of tetramer and intracellular interferon-γ staining. CMV pp65 peptides were used to stimulate T cells transduced with the HCV TCR (FIG. 15, lower panels) and untransduced cells (FIG. 15, upper panels). The T cells were stimulated overnight with 624 melanoma cells (FIG. 15, left panels), 624 melanoma cells expressing the HCV NS3:1406-1415 (FIG. 15, middle panels), or 624 melanoma cells expressing the CMV pp65:495-503 (FIG. 15, right panels). Cells were then stained with HLA-A2/CMV pp65:495-503 tetramers and counter stained for the presence of intracellular interferon-γ. Relative log fluorescence was measured by flow cytometry. The percentage of double stained cells is shown in the upper right quadrant of each histogram. Approximately 1% of the T cells in these TCR transduced T cell cultures express both TCRs which are capable of dual antigen recognition based on tetramer and intracellular IFN-γ staining. The results demonstrate that bifunctional T cells expressing both the HCV TCR as well as another TCR can be developed.

### Reference Example A

### Mouse Model of HCV+ Tumors

Two mouse strains will be used as models of HCV-positive tumors: rag-1^{-/-} mice on a C57BL6 background and HLA-A2-rag-1^{-/-} mice on a C57BL6 background will be made by crossing commercially available HLA-A2 transgenic mice to rag-1^{-/-} mice. To establish tumors, the mice will be injected either intravenously or subcutaneously with an HCV⁺ tumor cell line, such as Huh-7, HepG2, or human melanoma cell lines such as 624MEL. The cell lines will be transfected to express HLA-A2 as described by Nishimura et al., Cancer Research 59: 6230-6238 (1999). To confirm that the tumor cells maintain expression of HLA-A2 and HCV genes throughout the course of in vivo growth, tumors will be harvested, dissociated into a single cell suspension and stained with antibodies specific for HCV proteins and HLA-A2 and expression measured by FACS analysis.

Groups of twenty mice will be engrafted with 1×10⁵ to 1×10⁷ HCV TCR transduced T cells (CD8⁺ or a 1:1 ratio of CD8⁺ and CD4⁺ cells). Two mice from each group will be sacrificed on days 1, 2, 4, 7, 10, 14, 21, 30, 60, and 90 days post-infusion. At each time point, the total number of TCR gene modified T cells will be determined to assess persistence by counting the CD3⁺/CD34⁺ cells in major lymphoid compartments. Mice will be monitored and compared to those treated with T cells transduced with empty vector for signs of treatment related morbidity and mortality. Autopsies will be performed to look for subclinical signs of graft-versus-host disease (GVHD) or autoimmunity. In addition, recovered T cells will be analyzed in cytokine release assays to monitor T cell antigen reactivity and monitor development of immunologic memory by FACS analysis for expression of CD27, CD28, CD45RA and CCR7.

For tumor protection studies, groups of twenty rag-1^{-/-} mice will be injected in their tail veins with between 1×10⁵ and 1×10⁷ high or low affinity HCV TCR T cells. As controls, groups of twenty rag-1^{-/-} mice will receive no T cells or T cells transduced with the empty vector. The next day, five mice from each treatment group will receive an appropriate dose of Huh-7/A2 cells in their tail veins to establish lung metastases and five mice will receive an appropriate dose of Huh-7/A2 cells subcutaneously to establish solid tumors. The remaining ten mice from each treatment group will receive a similar number of Huh-7 cells intravenously or subcutaneously and will serve as specificity controls. Animals will be ear tagged and randomized to prevent investigator bias.

Mice bearing lung metastases will be sacrificed on day fourteen and the number of lung metastases will be counted. Mice with metastases too numerous to count will be considered to have 250 metastases for statistical analysis. Statistically significant differences in the mean number of lung metastases will be determined using the nonparametric two-tailed Kruskal-Wallis test. Mice bearing subcutaneous tumors will have their tumors measured using calipers daily until the control groups have tumors that are 1.25 cm in diameter. At that point, the experiment will be terminated and the remaining animals will be sacrificed. Statistically significant differences in tumor growth will be determined using the Wilcoxon Rank Sum test.

Each experiment will be repeated at least three times and the protective T cell dose, which is defined as the mean number of HCV TCR transduced T cells required to achieve a statistically significant protection from tumor challenge, will be determined. Statistically significant differences between the mean number of HCV TCR T cells versus TCR transduced TIL required for tumor protection will be determined using a one tailed T test.

For tumor treatment studies, mice bearing established Huh-7/A2 tumors will be engrafted with HCV TCR transduced T cells to determine if they can mediate regression of established 3 day lung metastases or subcutaneous solid tumors in vivo. Groups of forty rag-1^{-/-} mice will be injected in their tail veins with an appropriate dose of Huh-7/A2 or Huh-γ cells to establish lung metastases. Three days later, groups of five tumor bearing mice will be injected intravenously with between 1×10⁵ and 5×10⁷ high or low affinity HCV TCR transduced T cells. Five of the remaining mice will be injected with saline as a control for tumor growth and the other five will be injected with 5×10⁷ T cells transduced with the empty vector.

The mice bearing lung metastases will be sacrificed on day fourteen, ear tagged and randomized to prevent investigator bias, and the number of lung metastases will be counted. Mice with metastases too numerous to count will be considered to have 250 metastases for statistical analysis which will be completed as described above.

Mice bearing subcutaneous solid tumors will be established by injecting forty rag-1^{-/-} mice subcutaneously with an appropriate dose of Huh-7/A2 or Huh-7 cells. Once each tumor reaches approximately 0.5 cm in diameter, the mice will be injected intravenously with between 1×10⁵ and 5×10⁷ high or low affinity HCV TCR transduced T cells. Additional groups of five mice will receive no T cells to serve as an untreated control group or 5×10⁷ T cells transduced with the empty vector. All mice will then be ear tagged and randomized to prevent investigator bias during tumor measurements.

Tumor volume will be measured daily using calipers until the control groups have tumors that are 1.25 cm in diameter. At that point, the experiment will be terminated and the remaining animals will be sacrificed. Statistically significant differences in tumor growth will be determined using the Wilcoxon Rank Sum test as described above.

### Reference Example B

### Mouse Model of HCV Infection

A mouse model of HCV infection has previously been described by Mercer et al., Nat. Med. 7:927-933 (2001). Briefly, scid Alb/uPA mice will be transplanted with viable human hepatocytes within the first two weeks of life by intrasplenic inoculation. Mice with human α1-anti-trypsin (HAAT)>100 μg/L will be selected for inoculation with HCV. The HCV will be either a defined clone or human serum with high titer HCV. Two weeks post-infection, mouse serum will be assayed for HCV titer by real time PCR. Mice with HCV titers between 10⁴ and 10⁷ copies/mL will be used for further evaluation.

For virus protection studies, groups of five scid Alb/uPA mice engrafted with HLA-A2 hepatocytes will be injected in their tail veins initially with between 1×10⁵ and 5×10⁷ high or low affinity HCV TCR T cells or saline as a control. Twenty-four hours later, each mouse will be infected with HCV from the blood of HCV infected patients. Two weeks later and weekly thereafter up to eight weeks, blood from each mouse will be assayed in a blinded fashion for HAAT levels and for HCV titer by real time PCR to evaluate liver function and determine if the adoptive T cell transfer led to protection from HCV infection. At eight weeks post treatment, two animals from each group will be sacrificed and blood, spleen, liver, and lymph nodes will be collected to evaluate the HCV status of each animal and the persistence, localization, function and phenotype of the adoptively transferred T cells. Each experiment will be performed at least three times and statistically significant protection from HCV infection will be assessed using paired T tests.

For virus treatment studies, fifteen scid Alb/uPA mice engrafted with HLA-A2 hepatocytes will be infected with HCV virus from the blood of HCV infected patients. Two weeks later, an initial HCV titer will be measured in the blood of each mouse and groups of five of these HCV infected scid Alb/uPA-hep mice will be injected in their tails veins with the therapeutic dose of high or low affinity HCV TCR T cells or saline as a control. Blood will be obtained weekly for up to eight weeks and assayed in a blinded fashion for alanine aminotransferase (ALT) and human alpha1-antitrypsin (HAAT) levels and for HCV titer by real time PCR to evaluate liver function and determine if the adoptive T cell transfer led to protection from HCV infection. At eight weeks post treatment, two animals from each group will be sacrificed and blood, spleen, liver, and lymph nodes will be collected to evaluate the HCV status of each animal and the persistence, localization, function and phenotype of the adoptively transferred T cells. Each experiment will be performed at least three times and a statistically significant reduction of HCV titer will be assessed using paired T tests.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. If a concentration range is “at least 5%,” it is intended that all percentage values from at least 5% up to and including 100% are also expressly enumerated. These are only examples of what is specifically intended.

The invention has been described with reference to various specific embodiments and techniques. However, it should be understood that many variations and modifications may be made while remaining within the spirit and scope of the invention.

We claim:

1. A cell comprising an HCV epitope-reactive recombinant T cell receptor.

2. The cell of claim 1, wherein the HCV epitope is HLA-A2 restricted.

3. The cell of claim 1, wherein the HCV epitope comprises an NS3 epitope or a mutant thereof.

4. The cell of claim 3, wherein the NS3 epitope comprises NS3:1406-1415, or a mutant thereof.

5. The cell of claim 4, wherein the NS3 epitope comprises NS3:1406-1415 (V1408L), NS3:1406-1415 (A1409T), NS3:1406-1415 (I1412L), NS3:1406-1415 (I1412V), NS3:1406-1415 (I1412N), NS3:1406-1415 (V1408T) or NS3:1406-1415 (V1408S, A1409S, I1412L, A1414S).

6. The cell of claim 2, wherein the HCV epitope comprises a core protein epitope or a mutant thereof, an E1 epitope or a mutant thereof, an E2 epitope or a mutant thereof, a p7 epitope or a mutant thereof, an NS2 epitope or a mutant thereof; an NS4a epitope or a mutant thereof, an NS4b epitope or a mutant thereof, an NS5a epitope or a mutant thereof or an NS5b epitope or a mutant thereof.

7. The cell of claim 1, further comprising a second HCV epitope-reactive T cell receptor, wherein the second HCV epitope is different from the HCV epitope.

8. The cell of claim 1, wherein the T cell receptor comprises an α-chain having at least 95% amino acid identity to SEQ ID NO:2.

9. The cell of claim 1, wherein the TCR comprises a β-chain having at least 95% amino acid identity to SEQ ID NO:4.

10. The cell of claim 1, wherein the TCR comprises an α-chain having at least 95% amino acid identity to SEQ ID NO:2 and a β-chain having at least 95% amino acid identity to SEQ ID NO:4.

11. The cell of claim 1, further comprising a CD8 marker.

12. The cell of claim 1, further comprising a CD4 marker.

13. The cell of claim 1, wherein the cell is a hematopoietic stem cell.

14. The cell of claim 1, wherein the cell is a PBL-derived T cell.

15. An isolated polynucleotide comprising a sequence encoding an α-chain of an HCV epitope-reactive T cell receptor having at least 95% sequence identity to SEQ ID NO:2.

16. The polynucleotide of claim 15 comprising the sequence of SEQ ID NO:1.

17. An isolated polynucleotide comprising a sequence encoding a β-chain of a HCV epitope-reactive T cell receptor having at least 95% sequence identity to SEQ ID NO:4.

18. The polynucleotide of claim 17 comprising the sequence of SEQ ID NO:3.

19. An isolated polynucleotide comprising a sequence encoding an α-chain of an HCV epitope-reactive T cell receptor having at least 95% sequence identity to SEQ ID NO:2 and a sequence encoding a β-chain of an HCV epitope-reactive T cell receptor having at least 95% sequence identity to SEQ ID NO:4.

20. A construct comprising the polynucleotide of any of claims 15-19 operably connected to a promoter.

21. A retroviral vector comprising the construct of claim 20.

22. A T cell comprising the construct of claim 20.

23. A method of preparing an HCV-reactive T cell for delivery to a subject comprising transducing a T cell isolated from the subject with the construct of claim 20.

24. A method of preparing an HCV-reactive T cell receptor comprising:
(a) isolating an HCV-reactive T cell from a HCV⁺ recipient of an HLA mismatched liver allograft or from an HCV-exposed aviremic individual;
(b) cloning a polynucleotide sequence encoding the α- and β-chains of the T cell receptor from the HCV reactive T cell;
(c) delivering the polynucleotide sequence of step (b) to a cell; and
(d) incubating the cell under conditions suitable for expression of the T cell receptor by the cell.

25. A method of treating an HCV-infected subject or inhibiting reactivation of an HCV infection in a subject comprising administering to the subject an immunotherapeutically effective amount of cells comprising an HCV epitope-reactive recombinant T cell receptor.

26. The method of claim 25, wherein the T cell receptor comprises an α-chain having at least 95% amino acid identity to SEQ ID NO:2 and a β-chain having at least 95% amino acid identity to SEQ ID NO:4.

27. A method of treating HCV-related hepatocellular carcinoma in a subject comprising administering to the subject an immunotherapeutically effective amount of cells comprising an HCV epitope-reactive recombinant T cell receptor.

28. The method of claim 27, wherein the T cell receptor comprises an α-chain having at least 95% amino acid identity to SEQ ID NO:2 and a β-chain having at least 95% amino acid identity to SEQ ID NO:4.

29. Use of a cell comprising an HCV epitope-reactive recombinant T cell receptor in the preparation of a medicament for treating HCV infection or HCV-related hepatocellular carcinoma.

30. Use according to claim 29, wherein the HCV epitope is an NS3 epitope or mutant thereof.

31. Use according to claim 30, wherein the NS3 epitope comprises NS3:1406-1415.

32. Use according to claim 30, wherein the NS3 epitope comprises NS3:1406-1415 (V1408L), NS3:1406-1415 (I1412L), NS3:1406-1415 (I1412V), NS3:1406-1415 (I1412N) or NS3:1406-1415 (V1408T).

33. Use according to claim 29, wherein the T cell receptor comprises an α-chain having at least 95% amino acid identity to SEQ ID NO:2 and a β-chain having at least 95% amino acid identity to SEQ ID NO:4.


SEQUENCE LISTING (28 sequences)

SEQ ID NO: 1 - DNA, 87 bp, Homo sapiens
tatttctgtgcttatggagaagatgacaagatcatctttggaaaagggacacgacttcatattctccccaatatccagaaccctgac

SEQ ID NO: 2 - protein, 29 aa, Homo sapiens
YFCAYGEDDKIIFGKGTRLHILPNIQNPD

SEQ ID NO: 3 - DNA, 87 bp, Homo sapiens
tacctctgtgccagccggagggggccctacgagcagtacttcgggccgggcaccaggctcacggtcacagaggacctgaaaaacgtg

SEQ ID NO: 4 - protein, 29 aa, Homo sapiens
YLCASRRGPYEQYFGPGTRLTVTEDLKNV

SEQ ID NO: 5 - protein, 10 aa, Hepatitis C virus
YLLPRRGPRL

SEQ ID NO: 6 - protein, 10 aa, Hepatitis C virus
ADLMGYIPLV

SEQ ID NO: 7 - protein, 9 aa, Hepatitis C virus
CINGVCWTV

SEQ ID NO: 8 - protein, 10 aa, Hepatitis C virus
KLVALGINAV

SEQ ID NO: 9 - protein, 10 aa, Hepatitis C virus
KLLALGINAV

SEQ ID NO: 10 - protein, 10 aa, Hepatitis C virus
KLVTLGINAV

SEQ ID NO: 11 - protein, 10 aa, Hepatitis C virus
KLVALGLNAV

SEQ ID NO: 12 - protein, 10 aa, Hepatitis C virus
KLVALGVNAV

SEQ ID NO: 13 - protein, 10 aa, Hepatitis C virus
KLVALGNNAV

SEQ ID NO: 14 - protein, 10 aa, Hepatitis C virus
KLTALGINAV

SEQ ID NO: 15 - protein, 10 aa, Hepatitis C virus
KLSSLGLNSV

SEQ ID NO: 16 - protein, 9 aa, Hepatitis C virus
ALYDVVTKL

SEQ ID NO: 17 - DNA, 38 bp, Homo sapiens
aaagtcgacctgtgagcatggcatgccctggcttcctg

SEQ ID NO: 18 - DNA, 35 bp, Homo sapiens
aaagtcgactaataatggtgaagatccggcaattt

SEQ ID NO: 19 - DNA, 45 bp, Homo sapiens
aaagtcgaccctcagctggaccacagccgcagcgtcatgagcaga

SEQ ID NO: 20 - DNA, 36 bp, Homo sapiens
aaactcgagccccaactgtgccatgactatcaggct

SEQ ID NO: 21 - DNA, 45 bp, Homo sapiens
aaactcgagctagcctctggaatcctttctcttgaccattgccat

SEQ ID NO: 22 - protein, 9 aa, Cytomegalovirus
NLVPMVATV

SEQ ID NO: 23 - protein, 9 aa, Homo sapiens
YMDGTMSQV

SEQ ID NO: 24 - DNA, 34 bp, Homo sapiens
aaactcgagcgcgtcatggccttaccagtgaccg

SEQ ID NO: 25 - DNA, 30 bp, Homo sapiens
aaactcgagttagacgtatctcgccgaaag

SEQ ID NO: 26 - DNA, 35 bp, Homo sapiens
aaagtcgacgccacgatgcggccgcggctgtggct

SEQ ID NO: 27 - DNA, 30 bp, Homo sapiens
gtcgacaataaacacttcaacaaagcactc

SEQ ID NO: 28 - protein, 10 aa, Hepatitis C virus
KLSGLGLNAV